(19) United States
(12) Patent Application Publication
(10) Pub. No.: US 2014/0037604 A1
(43) Pub. Date: Feb. 6, 2014

(54) NUTRITIONAL COMPOSITIONS COMPRISING ALPHA-HYDROXYISOCAPROIC ACID

(71) Applicants: Norman Alan Greenberg, New Hope, MN (US); Denis Breuille, Lausanne (CH); Zamzam Kabiry (Fariba) Roughead, Plymouth, MN (US); Doug Bolster, Lake in the Hills, IL (US); Jennifer Mager, St. Louis, MO (US)

(72) Inventors: Norman Alan Greenberg, New Hope, MN (US); Denis Breuille, Lausanne (CH); Zamzam Kabiry (Fariba) Roughead, Plymouth, MN (US); Doug Bolster, Lake in the Hills, IL (US); Jennifer Mager, St. Louis, MO (US)

(73) Assignee: NESTEC S.A., Vevey (CH)

(21) Appl. No.: 14/112,744

(22) PCT Filed: Apr. 18, 2012
(86) PCT No.: PCT/EP2012/057092

§ 371 Date: Oct. 18, 2013

Related U.S. Application Data
(60) Provisional application No. 61/476,345, filed on Apr. 18, 2011.

(51) Int. Cl. A61K 31/19; A23L 1/30; A61K 45/06

(52) U.S. Cl. CPC A61K 31/19; A61K 45/06; A23L 1/30
USPC 424/93.41; 514/557; 424/93.44; 424/93.5; 424/93.4; 424/93.45; 424/93.51; 424/93.42; 424/94.1; 514/44 R

(57) ABSTRACT

Nutritional compositions for maximizing muscle protein synthesis while minimizing the catabolism of muscle proteins and methods of using same are provided. In this manner, the nutritional compositions may provide for retention of lean body mass, which helps to avoid loss of independence and functionality, as well as to improve quality of life especially in the elderly at risk of sarcopenia and frailty. The nutritional compositions include α-hydroxyisocaproic acid and may include other functional ingredients such as, but not limited to whey protein including whey protein micelles, prebiotic fibers, L-carnitine, nucleotides, and amino acids. Methods of administering such nutritional products to individuals in need of same are also provided.

### BACKGROUND

The present disclosure relates generally to health and nutrition. More specifically, the present disclosure relates to nutritional compositions having α-hydroxyisocaproic acid and derivatives and methods of using same.

There are many types of nutritional compositions currently on the market. Nutritional compositions can be targeted toward certain consumer types, for example, young, elderly, athletic, etc., based on the specific ingredients of the nutritional composition. For example, the elderly and individuals with certain illnesses can often times experience a reduction in lean body mass that is due, at least in part, to a reduction in muscle protein synthesis (such as sarconpenia), reduced intake, or increased demand due to illness or presence of inflammation. A reduction in lean body mass can lead to the loss of metabolic stability (glucose tolerance, insulin sensitivity), independence, functionality, and quality of life, as well as a decline in cognitive ability. These individuals, therefore, would benefit significantly by administration of a diet directed to maximizing the anabolism and minimizing the catabolism of muscle tissue. The diets could also provide further benefits to the individuals by combining into a nutritional composition different types of functional compounds, which provide different types of physiological advantages.

One goal of nutritional support, therefore, is to provide individuals requiring improved muscle performance and/or maintenance of lean body mass and muscle strength/power with nutritional compositions that provide physiological benefits with respect to same.

### SUMMARY

The present disclosure is directed to nutritional compositions having α-hydroxyisocaproic acid and methods of using same. In an embodiment, the nutritional compositions include an effective amount of α-hydroxyisocaproic acid. In another embodiment, the nutritional compositions include an effective amount of α-hydroxyisocaproic acid and an effective amount of citrulline. In yet another embodiment, the nutritional compositions include an effective amount of α-hydroxyisocaproic acid and an effective amount of α-ketoglutarate. In still yet another embodiment, the nutritional compositions include an effective amount of α-hydroxyisocaproic acid and eicosapentaenoic acid.

In an embodiment, the α-hydroxyisocaproic acid is present in an amount from about 0.15 to about 10 g, preferably from about 2 g to about 10 g. The α-hydroxyisocaproic acid may also be present in an amount of about 0.5 g to about 5 g, more preferably from about 2 g to 5 g, most preferably about 1.5 g.

In an embodiment, the nutritional compositions include a source of ω-3 fatty acids, wherein the source of ω-3 fatty acids is selected from the group consisting of fish oil, krill, plant sources containing ω-3 fatty acids, flaxseed, canola oil, walnut, algae, or combinations thereof. The ω-3 fatty acids are selected from the group consisting of α-linolenic acid (“ALA”), stearidonic acid (SDA), docosahexaenoic acid (“DHA”), eicosapentaenoic acid (“EPA”), or combinations thereof.

In an embodiment, the nutritional compositions include at least one nucleotide selected from the group consisting of a subunit of deoxyribonucleic acid (“DNA”), a subunit of ribonucleic acid (“RNA”), polymeric forms of DNA and RNA, yeast RNA, or combinations thereof. The at least one nucleotide may be an exogenous nucleotide. The nucleotide may be provided in an amount of about 0.5 g to 3 g per day.

In an embodiment, the nutritional compositions include a phytonutrient selected from the group consisting of flavanoids, allied phenolic compounds, polyphenolic compounds, terpenoids, alkaloids, sulphur-containing compounds, or combinations thereof. The phytonutrient may be selected from the group consisting of carotenoids, plant sterols, quercetin, curcumin, limonin, or combinations thereof.

In an embodiment, the nutritional compositions include a source of protein. The source of protein may provide the nutritional composition with at least 10 g of high quality protein or to provide an amount of protein of at least 10 g per day. The source of protein may be selected from the group consisting of dairy based proteins, plant based proteins, animal based proteins, artificial proteins, or combinations thereof. The dairy based proteins may be selected from the group consisting of casein, micellar casein, caseinates, casein hydrolysate, whey, whey hydrolysates, whey concentrates, whey isolates, milk protein concentrate, milk protein isolate, or combinations thereof. The plant based proteins are selected from the group consisting of soy protein, pea protein, canola protein, wheat and fractionated wheat proteins, corn proteins, zein proteins, rice proteins, oat proteins, potato proteins, peanut proteins, green pea powder, green bean powder, spirulina, proteins derived from vegetables, beans, buckwheat, lentils, pulses, single cell proteins, or combinations thereof.

In an embodiment, the nutritional compositions include a prebiotic selected from the group consisting of acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, or combinations thereof.

In an embodiment, the nutritional compositions include a probiotic selected from the group consisting of Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, non-replicating microorganisms, or combinations thereof.

In an embodiment, the nutritional compositions include an amino acid selected from the group consisting of alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, ornithine, tryptophan, tyrosine, valine, or combinations thereof. In an embodiment, the amino acid is citrulline. In an embodiment, the amino acid is a branched chain amino acid selected from the group consisting of isoleucine, leucine, valine, or combinations thereof.

In an embodiment, the nutritional compositions include an antioxidant selected from the group consisting of astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, or combinations thereof.

In an embodiment, the nutritional compositions include a vitamin selected from the group consisting of vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e., MK-4, MK-7), folic acid, biotin, choline or combinations thereof.

In an embodiment, the nutritional compositions include a mineral selected from the group consisting of boron, calcium, chromium, copper, iodine, iron, magnesium, manganese, molybdenum, nickel, phosphorus, potassium, selenium, silicon, tin, vanadium, zinc, or combinations thereof.

In an embodiment, the nutritional compositions include a compound selected from the group consisting of α-ketoglutarate, L-carnitine, or combinations thereof.

In an embodiment, the nutritional compositions are in a form selected from the group consisting of tablets, capsules, liquids, chewables, soft gels, sachets, powders, syrups, liquid suspensions, emulsions, solutions, or combinations thereof. In an embodiment, the nutritional compositions are in the form of a powder.

In an embodiment, the nutritional compositions are oral nutritional supplements, a tube feeding, or combinations thereof.

In an embodiment, the nutritional compositions are a source of complete nutrition. In another embodiment, the nutritional compositions are a source of incomplete nutrition.

In yet another embodiment, methods for stimulating muscle protein synthesis in an individual in need of same are provided. The methods include administering to the individual a nutritional composition comprising an effective amount of α-hydroxyisocaproic acid.

In still yet another embodiment, methods for minimizing catabolism of muscle protein in an individual in need of same are provided. The methods include administering to the individual a nutritional composition comprising an effective amount of α-hydroxyisocaproic acid.

In another embodiment, methods for preserving lean body mass in an individual in need of same are provided. The methods include administering to the individual a nutritional composition comprising an effective amount of α-hydroxyisocaproic acid.

In yet another embodiment, methods for reducing unloading-induced bone loss in an individual in need of same are provided. The methods include administering to the individual a nutritional composition comprising an effective amount of α-hydroxyisocaproic acid.

In still yet another embodiment, methods for attenuating skeletal muscle atrophy in an individual in need of same are provided. The methods include administering to the individual a nutritional composition comprising an effective amount of α-hydroxyisocaproic acid.

In another embodiment, methods for alleviating a high uremic load in an individual in need of same are provided. The methods include administering to the individual a nutritional composition comprising an effective amount of α-hydroxyisocaproic acid.

In an embodiment, the individual is selected from the group consisting of the elderly, those with a medical condition, or combinations thereof.

In an embodiment, the nutritional compositions are administered to the individual so as to provide the individual with about 0.15 g to about 10 g per day, preferably from about 2 g to about 10 g per day, more preferably from about 0.5 g to about 5 g, more preferably from about 150 mg to about 2.5 g of α-hydroxyisocaproic acid per day. The nutritional compositions may also be administered to the individual so as to provide the individual with about 0.5 g to about 5 g per day, more preferably from about 2 g to 5 g, or preferably about 1.5 g per day.

In an embodiment, the nutritional compositions further include citrulline. The nutritional compositions may be administered to the individual so as to provide the individual with about 1 g to about 15 g citrulline per day, more preferably from about 2 g to about 15 g of citrulline per day, even more preferably from about 2 g to about 7 g, even more preferably from about 2 g to about 5 g of citrulline per day. The nutritional compositions may also be administered to the individual so as to provide the individual with about 4 g to about 7 g of citrulline per day.

In an embodiment, the nutritional compositions further include α-ketoglutarate in a form selected from the group consisting of ornithine α-ketoglutarate, arginine α-ketoglutarate, ketoisocaproic acid (KIC) or combinations thereof. The nutritional compositions may be administered to the individual so as to provide the individual with about 2 g to about 20 g of α-ketoglutarate per day. The nutritional composition may also be administered to the individual so as to provide the individual with about 10 g to about 30 g of α-ketoglutarate per day.

In an embodiment, the nutritional compositions further include eicosapentaenoic acid. The nutritional compositions may be administered to the individual so as to provide the individual with about 0.25 g to about 5 g, more preferably form about 250 mg to about 3 g, even more preferably from about 250 mg to 1.5 g of eicosapentaenoic acid per day.

In an embodiment, the nutritional compositions further include at least one nucleotide selected from the group consisting of a subunit of deoxyribonucleic acid (“DNA”), a subunit of ribonucleic acid (“RNA”), polymeric forms of DNA and RNA, yeast RNA, or combinations thereof. The at least one nucleotide may be an exogenous nucleotide.

In an embodiment, the nutritional compositions further include at least one branched chain amino acid selected from the group consisting of leucine, isoleucine, valine, or combinations thereof.

In an embodiment, the nutritional compositions further include L-carnitine.

An advantage of the present disclosure is to provide improved nutritional compositions.

Another advantage of the present disclosure is to provide nutritional compositions that maximize skeletal muscle protein synthesis.

Another advantage of the present disclosure is to provide nutritional compositions that minimize catabolism of skeletal muscle proteins.

Yet another advantage of the present disclosure is to provide nutritional compositions that preserve lean body mass.

Still yet another advantage of the present disclosure is to provide nutritional compositions that help to improve recovery from physical activity.

Another advantage of the present disclosure is to provide nutritional compositions that help to reduce healthcare costs.

Yet another advantage of the present disclosure is to provide nutritional compositions that help to reduce unloading-induced bone loss.

Still yet another advantage of the present disclosure is to provide nutritional compositions that help to attenuate skeletal muscle atrophy in individuals in need of same.

Another advantage of the present disclosure is to provide nutritional compositions that help to alleviate a high uremic load.

Additional features and advantages are described herein, and will be apparent from the following Detailed Description.

### DETAILED DESCRIPTION

As used herein, “about” is understood to refer to numbers in a range of numerals. Moreover, all numerical ranges herein should be understood to include all integer, whole or fractions, within the range.

As used herein the term α-hydroxyisocaproic acid is understood as also comprising analogs of α-hydroxyisocaproic acid such as keto-isocaproic acid (KIC), for example.

As used herein the term “amino acid” is understood to include one or more amino acids. The amino acid can be, for example, alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, ornithine or combinations thereof.

As used herein, “animal” includes, but is not limited to, mammals, which include but is not limited to, rodents, aquatic mammals, domestic animals such as dogs and cats, farm animals such as sheep, pigs, cows and horses, and humans. Wherein the terms “animal” or “mammal” or their plurals are used, it is contemplated that it also applies to any animals that are capable of the effect exhibited or intended to be exhibited by the context of the passage.

As used herein, the term “antioxidant” is understood to include any one or more of various substances such as beta-carotene (a vitamin A precursor), vitamin C, vitamin E, and selenium) that inhibit oxidation or reactions promoted by Reactive Oxygen Species (“ROS”) and other radical and non-radical species. Additionally, antioxidants are molecules capable of slowing or preventing the oxidation of other molecules. Non-limiting examples of antioxidants include astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, or combinations thereof.

As used herein, “complete nutrition” includes nutritional products and compositions that contain sufficient types and levels of macronutrients (protein, fats and carbohydrates) and micronutrients to be sufficient to be a sole source of nutrition for the animal to which it is being administered to. Patients can receive 100% of their nutritional requirements from such complete nutritional compositions.

As used herein, “effective amount” is an amount that prevents a deficiency, treats a disease or medical condition in an individual or, more generally, reduces symptoms, manages progression of the diseases or provides a nutritional, physiological, or medical benefit to the individual. A treatment can be patient- or doctor-related.

While the terms “individual” and “patient” are often used herein to refer to a human, the invention is not so limited. Accordingly, the terms “individual” and “patient” refer to any animal, mammal or human having or at risk for a medical condition that can benefit from the treatment.

As used herein, sources of ω-3 fatty acids include, for example, fish oil, krill, plant sources of ω-3, flaxseed, canola oil, walnut, and algae. Examples of ω-3 fatty acids include, for example, α-linolenic acid (“ALA”), docosahexaenoic acid (“DHA”), stearidonic acid (SDA), eicosapentaenoic acid (“EPA”), or combinations thereof.

As used herein, “food grade micro-organisms” means micro-organisms that are used and generally regarded as safe for use in food.

As used herein, “incomplete nutrition” includes nutritional products or compositions that do not contain sufficient levels of macronutrients (protein, fats and carbohydrates) or micronutrients to be sufficient to be a sole source of nutrition for the animal to which it is being administered to. Partial or incomplete nutritional compositions can be used as a nutritional supplement.

As used herein, “long term administrations” are preferably continuous administrations for more than 6 weeks. Alternatively, “short term administrations,” as used herein, are continuous administrations for less than 6 weeks.

As used herein, “mammal” includes, but is not limited to, rodents, aquatic mammals, domestic animals such as dogs and cats, farm animals such as sheep, pigs, cows and horses, and humans. Wherein the term “mammal” is used, it is contemplated that it also applies to other animals that are capable of the effect exhibited or intended to be exhibited by the mammal.

The term “microorganism” is meant to include the bacterium, yeast and/or fungi, a cell growth medium with the microorganism, or a cell growth medium in which microorganism was cultivated.

As used herein, the term “minerals” is understood to include boron, calcium, chromium, copper, iodine, iron, magnesium, manganese, molybdenum, nickel, phosphorus, potassium, selenium, silicon, tin, vanadium, zinc, or combinations thereof.

As used herein, a “non-replicating” microorganism means that no viable cells and/or colony forming units can be detected by classical plating methods. Such classical plating methods are summarized in the microbiology book: James Monroe Jay, et al., “Modern food microbiology,” 7th edition, Springer Science, New York, N.Y. p. 790 (2005). Typically, the absence of viable cells can be shown as follows: no visible colony on agar plates or no increasing turbidity in liquid growth medium after inoculation with different concentrations of bacterial preparations (‘non replicating’ samples) and incubation under appropriate conditions (aerobic and/or anaerobic atmosphere for at least 24 h). For example, bifidobacteria such as Bifidobacterium longum, Bifidobacterium lactis and Bifidobacterium breve or lactobacilli, such as Lactobacillus paracasei or Lactobacillus rhamnosus, may be rendered non-replicating by heat treatment, in particular low temperature/long time heat treatment.

As used herein, “normal bone growth” refers to the process by which childhood and adolescent bones are sculpted by modeling, which allows for the formation of new bone at one site and the removal of old bone from another site within the same bone. This process allows individual bones to grow in size and to shift in space. During childhood bones grow because resorption (the process of breaking down bone) occurs inside the bone while formation of new bone occurs on its outer (periosteal) surface. At puberty the bones get thicker because formation can occur on both the outer and inner (endosteal) surfaces. The remodeling process occurs throughout life and becomes the dominant process by the time that bone reaches its peak mass (typically by the early 20s). In remodeling, a small amount of bone on the surface of trabeculae or in the interior of the cortex is removed and then replaced at the same site. The remodeling process does not change the shape of the bone, but it is nevertheless vital for bone health. Modeling and remodeling continue throughout life so that most of the adult skeleton is replaced about every 10 years. While remodeling predominates by early adulthood, modeling can still occur particularly in response to weakening of the bone.

As used herein, a “nucleotide” is understood to be a subunit of deoxyribonucleic acid (“DNA”), ribonucleic acid (“RNA”), polymeric RNA, polymeric DNA, or combinations thereof. It is an organic compound made up of a nitrogenous base, a phosphate molecule, and a sugar molecule (deoxyribose in DNA and ribose in RNA). Individual nucleotide monomers (single units) are linked together to form polymers, or long chains. Exogenous nucleotides are specifically provided by dietary supplementation. The exogenous nucleotide can be in a monomeric form such as, for example, 5 ‘-Adenosine Monophosphate (“5’-AMP”), 5′-Guanosine Monophosphate (“5′-GMP”), 5′-Cytosine Monophosphate (“5′-CMP”), 5′-Uracil Monophosphate (“5′-UMP”), 5′-Inosine Monophosphate (“5′-IMP”), 5′-Thymine Monophosphate (“5′-TMP”), or combinations thereof. The exogenous nucleotide can also be in a polymeric form such as, for example, an intact RNA. There can be multiple sources of the polymeric form such as, for example, yeast RNA.

“Nutritional products,” or “nutritional compositions,” as used herein, are understood to include any number of optional additional ingredients, including conventional food additives (synthetic or natural), for example one or more acidulants, additional thickeners, buffers or agents for pH adjustment, chelating agents, colorants, emulsifies, excipient, flavor agent, mineral, osmotic agents, a pharmaceutically acceptable carrier, preservatives, stabilizers, sugar, sweeteners, texturizers, and/or vitamins. The optional ingredients can be added in any suitable amount. The nutritional products or compositions may be a source of complete nutrition or may be a source of incomplete nutrition.

As used herein the term “patient” is understood to include an animal, especially a mammal, and more especially a human that is receiving or intended to receive treatment, as it is herein defined.

As used herein, “phytochemicals” or “phytonutrients” are non-nutritive compounds that are found in many foods. Phytochemicals are functional foods that have health benefits beyond basic nutrition, are health promoting compounds that come from plant sources, and may be natural or purified. “Phytochemicals” and “Phytonutrients” refers to any chemical produced by a plant that imparts one or more health benefit on the user. Non-limiting examples of phytochemicals and phytonutrients include those that are:

i) phenolic compounds which include monophenols (such as, for example, apiole, carnosol, carvacrol, dillapiole, rosemarinol); flavonoids (polyphenols) including flavonols (such as, for example, quercetin, fingerol, kaempferol, myricetin, rutin, isorhamnetin), flavanones (such as, for example, fesperidin, naringenin, silybin, eriodictyol), flavones (such as, for example, apigenin, tangeritin, luteolin), flavan-3-ols (such as, for example, catechins, (+)-catechin, (+)-gallocatechin, (−)-epicatechin, (−)-epigallocatechin, (−)-epigallocatechin gallate (EGCG), (−)-epicatechin 3-gallate, theaflavin, theaflavin-3-gallate, theaflavin-3′-gallate, theaflavin-3,3′-digallate, thearubigins), anthocyanins (flavonals) and anthocyanidins (such as, for example, pelargonidin, peonidin, cyanidin, delphinidin, malvidin, petunidin), isoflavones (phytoestrogens) (such as, for example, daidzein (formononetin), genistein (biochanin A), glycitein), dihydroflavonols, chalcones, coumestans (phytoestrogens), and Coumestrol; Phenolic acids (such as: Ellagic acid, Gallic acid, Tannic acid, Vanillin, curcumin); hydroxycinnamic acids (such as, for example, caffeic acid, chlorogenic acid, cinnamic acid, ferulic acid, coumarin); lignans (phytoestrogens), silymarin, secoisolariciresinol, pinoresinol and lariciresinol); tyrosol esters (such as, for example, tyrosol, hydroxytyrosol, oleocanthal, oleuropein); stilbenoids (such as, for example, resveratrol, pterostilbene, piceatannol) and punicalagins;

ii) terpenes (isoprenoids) which include carotenoids (tetraterpenoids) including carotenes (such as, for example, α-carotene, β-carotene, γ-carotene, δ-carotene, lycopene, neurosporene, phytofluene, phytoene), and xanthophylls (such as, for example, canthaxanthin, cryptoxanthin, aeaxanthin, astaxanthin, lutein, rubixanthin); monoterpenes (such as, for example, limonene, perillyl alcohol); saponins; lipids including: phytosterols (such as, for example, campesterol, beta sitosterol, gamma sitosterol, stigmasterol), tocopherols (vitamin E), and omega-3, 6, and 9 fatty acids (such as, for example, gamma-linolenic acid); triterpenoid (such as, for example, oleanolic acid, ursolic acid, betulinic acid, moronic acid);

iii) betalains which include Betacyanins (such as: betanin, isobetanin, probetanin, neobetanin); and betaxanthins (non glycosidic versions) (such as, for example, indicaxanthin, and vulgaxanthin);

iv) organosulfides, which include, for example, dithiolthiones (isothiocyanates) (such as, for example, sulphoraphane); and thiosulphonates (allium compounds) (such as, for example, allyl methyl trisulfide, and diallyl sulfide), indoles, glucosinolates, which include, for example, indole-3-carbinol; sulforaphane; 3,3′-diindolylmethane; sinigrin; allicin; alliin; allyl isothiocyanate; piperine; syn-propanethial-S-oxide;

v) protein inhibitors, which include, for example, protease inhibitors;

vi) other organic acids which include oxalic acid, phytic acid (inositol hexaphosphate); tartaric acid; and anacardic acid; or

vii) combinations thereof.

As used in this disclosure and the appended claims, the singular forms “a,” “an” and “the” include plural referents unless the context clearly dictates otherwise. Thus, for example, reference to “a polypeptide” includes a mixture of two or more polypeptides, and the like.

As used herein, a “prebiotic” is a food substance that selectively promotes the growth of beneficial bacteria or inhibits the growth or mucosal adhesion of pathogenic bacteria in the intestines. They are not inactivated in the stomach and/or upper intestine or absorbed in the gastrointestinal tract of the person ingesting them, but they are fermented by the gastrointestinal microflora and/or by probiotics. Prebiotics are, for example, defined by Glenn R. Gibson and Marcel B. Roberfroid, “Dietary Modulation of the Human Colonic Microbiota: Introducing the Concept of Prebiotics,” J. Nutr., 125: 1401-1412 (1995). Non-limiting examples of prebiotics include acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, or their hydrolysates, or combinations thereof.

As used herein, probiotic micro-organisms (hereinafter “probiotics”) are food-grade microorganisms (alive, including semi-viable or weakened, and/or non-replicating), metabolites, microbial cell preparations or components of microbial cells that could confer health benefits on the host when administered in adequate amounts, more specifically, that beneficially affect a host by improving its intestinal microbial balance, leading to effects on the health or well-being of the host. See, Salminen S, Ouwehand A. Benno Y. et al., “Probiotics: how should they be defined?,” Trends Food Sci. Technol., 10, 107-10 (1999). In general, it is believed that these micro-organisms inhibit or influence the growth and/or metabolism of pathogenic bacteria in the intestinal tract. The probiotics may also activate the immune function of the host. For this reason, there have been many different approaches to include probiotics into food products. Non-limiting examples of probiotics include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, or combinations thereof.

The terms “protein,” “peptide,” “oligopeptides” or “polypeptide,” as used herein, are understood to refer to any composition that includes, a single amino acids (monomers), two or more amino acids joined together by a peptide bond (dipeptide, tripeptide, or polypeptide), collagen, precursor, homolog, analog, mimetic, salt, prodrug, metabolite, or fragment thereof or combinations thereof. For the sake of clarity, the use of any of the above terms is interchangeable unless otherwise specified. It will be appreciated that polypeptides (or peptides or proteins or oligopeptides) often contain amino acids other than the 20 amino acids commonly referred to as the 20 naturally occurring amino acids, and that many amino acids, including the terminal amino acids, may be modified in a given polypeptide, either by natural processes such as glycosylation and other post-translational modifications, or by chemical modification techniques which are well known in the art. Among the known modifications which may be present in polypeptides of the present invention include, but are not limited to, acetylation, acylation, ADP-ribosylation, amidation, covalent attachment of a flavanoid or a heme moiety, covalent attachment of a polynucleotide or polynucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycation, glycosylation, glycosylphosphatidyl inositol (“GPI”) membrane anchor formation, hydroxylation, iodination, methylation, myristoylation, oxidation, proteolytic processing, phosphorylation, prenylation, racemization, selenoylation, sulfation, transfer-RNA mediated addition of amino acids to polypeptides such as arginylation, and ubiquitination. The term “protein” also includes “artificial proteins” which refers to linear or non-linear polypeptides, consisting of alternating repeats of a peptide.

Non-limiting examples of proteins include dairy based proteins, plant based proteins, animal based proteins and artificial proteins. Dairy based proteins may be selected from the group consisting of casein, micellar casein, caseinates, casein hydrolysate, whey, whey hydrolysates, whey concentrates, whey isolates, milk protein concentrate, milk protein isolate, or combinations thereof. Plant based proteins include, for example, soy protein (e.g., all forms including concentrate and isolate), pea protein (e.g., all forms including concentrate and isolate), canola protein (e.g., all forms including concentrate and isolate), other plant proteins that commercially are wheat and fractionated wheat proteins, corn and it fractions including zein, rice, oat, potato, peanut, and any proteins derived from beans, buckwheat, lentils, pulses, single cell proteins, or combinations thereof. Animal based proteins may be selected from the group consisting of beef, poultry, fish, lamb, seafood, or combinations thereof.

All dosage ranges contained within this application are intended to include all numbers, whole or fractions, contained within said range.

As used herein, a “synbiotic” is a supplement that contains both a prebiotic and a probiotic that work together to improve the microflora of the intestine.

As used herein, the terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition, such as nitrogen imbalance or muscle loss. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition.

As used herein, a “tube feed” is a complete or incomplete nutritional product or composition that is administered to an animal's gastrointestinal system, other than through oral administration, including but not limited to a nasogastric tube, orogastric tube, gastric tube, jejunostomy tube (“J-tube”), percutaneous endoscopic gastrostomy (“PEG”), port, such as a chest wall port that provides access to the stomach, jejunum and other suitable access ports.

As used herein the term “vitamin” is understood to include any of various fat-soluble or water-soluble organic substances (non-limiting examples include vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e. MK-4, MK-7), folic acid and biotin) essential in minute amounts for normal growth and activity of the body and obtained naturally from plant and animal foods or synthetically made, pro-vitamins, derivatives, choline, analogs.

The present disclosure is related to nutritional compositions having a combination of nutrients and food ingredients to maximize muscle protein synthesis while minimizing the catabolism of muscle proteins such that the lean body mass of patients including, for example, the elderly and those with illness is preserved as well as possible.

The nutritional compositions of the present disclosure include α-hydroxycaproic acid (“HICA”) in combination with other compounds to maximize the anabolism and minimize the catabolism of muscle tissue. Applicant has found that various combinations with α-HICA deliver superior benefits due to better taste profile (improving compliance and therefore efficacy) as well as complementary metabolic benefits. For example, α-HICA is a leucine metabolite with anabolic benefits directly related to protein synthesis while other compounds such as citrulline deliver benefits ancillary to the anabolic process.

The translational control of skeletal muscle protein synthesis includes control points at initiation, elongation and termination. In addition to the step in translation initiation involving the binding of messenger ribonucleic acid (“mRNA”) to the 40S ribosomal subunit, regulation can occur through modulation of the binding of the initiator methionyl-tRNA (“met-tRNAi”) to the 40S ribosomal subunit to form the 43 S preinitiation complex. In this step, the eIF2-GTP-met-tRNAi complex binds to the 40S ribosomal subunit to form a ternary complex. The guanosine triphosphate (“GTP”) bound to eIF2 is subsequently hydrolyzed to guanosine diphosphate (“GDP”), and the eIF2-GDP complex is released from the 40S ribosomal subunit. eIF2 must then exchange GDP for GTP in order to participate in a subsequent round of initiation and form a new ternary complex. A second translation initiation factor, eIF2B, mediates the guanine nucleotide exchange on eIF2 and the inhibition of eIF2B activity reduces the amount of eIF2-GTP available for ternary complex formation. In part, the activity of eIF2B is regulated by phosphorylation of the α-subunit of eIF2, which becomes a competitive inhibitor of eIF2B when phosphorylated on the α-subunit. Moreover, α-HICA mediates its acute effects on global protein synthesis via enhanced translational efficiency through increased eIF2B activity and ternary complex formation.

The nutritional compositions of the present disclosure may be provided to an individual or patient in one bolus, or in several smaller doses. However, the nutritional compositions of the present disclosure should provide the individual with an amount of α-HICA ranging from about 0.15 to about 10 g per day, preferably from about 2 g to about 10 g per day, more preferably from about 150 mg to about 2.5 g of α-hydroxyisocaproic acid per day. In an embodiment, the individual is provided with about 0.5 g to about 5 g per day, more preferably from about 2 g to 5 g, even more preferably 1.5 g α-HICA per day.

In an embodiment, the nutritional compositions are administered to the individual so as to provide the individual with about The nutritional compositions may also be administered to the individual so as to provide the individual with about

In an embodiment, nutritional compositions of the present disclosure include α-HICA and citrulline. Citrulline is a non-protein amino acid that is found in significant dietary amounts only in watermelon (Citrullus lanatus). Intake of citrulline can lead to formation of polyamines. Polyamines such as agmatine, putrescine, spermidine and spermine have been reported to be involved in a variety of physiological and biochemical phenomena including upregulation of protein kinase C (“PKC”), extracellular signal-regulated kinase (“ERK”), and transforming growth factor-beta1 (“TGF-beta1”).

The metabolic fate of citrulline is conversion to arginine. In fact, citrulline is very effective in raising serum arginine, which is a source of nitric oxide (“NO”) in the body. NO is important for relaxation of blood vessels and delivery of blood flow to tissues in the body. With improved blood flow, nutrients and other compounds in the blood can be delivered more efficiently to the skeletal muscle tissues. Further, NO is an anabolic signal as well as a facilitator for stimulation of protein synthesis and release of growth factors such as polyamines mentioned above. NO also leads to release of insulin and IGF-1 leading to increased uptake of anabolic substrates as well as bio-utlization of the substrates.

Guadagni and Biolo indicate that additional protein may be needed in individuals with inflammation (such as the elderly or individuals with illness) in part to maintain the levels of arginine and glutamine. See, Guadagni and Biolo, Effects of inflammation and/or inactivity on the need for dietary protein, Volume 12, Issue 6, p. 617-622 (2009). Citrulline can serve to maintain arginine levels. Additionally, it can help to maintain glutamine levels since glutamine conversion to citrulline in the small intestine will be reduced by a feedback signal from the citrulline provided exogenously. This will reduce the need for muscle catabolism to provide arginine and glutamine for bodily functions.

It is further possible that the combination of α-HICA and citrulline will synergistically improve the maintenance of lean body mass in elderly that do a limited amount of exercise and/or physical therapy. Citrulline has been shown to have an anabolic effect in malnourished aged animals. The anabolic signal in the elderly population is typically down-regulated. The addition of both α-HICA and citrulline will provide a strong boost to this signal. This improved recovery from physical activity will allow for accelerated recovery from forced inactivity due to illness or trauma. A reduction in cost of care could also be realized based on reduced number physical therapy sessions and a faster return to full independent living and a return to work.

As mentioned above, the nutritional compositions of the present disclosure may be provided to an individual or patient in one bolus, or in several smaller doses. However, the nutritional compositions of the present disclosure should provide the individual with an amount of citrulline ranging from about 1 g to about 15 g citrulline per day, more preferably from about 2 g to about 15 g of citrulline per day, even more preferably from about 2 g to about 7 g, even more preferably from about 2 g to about 5 g of citrulline per day. In an embodiment, the individual is provided with from about 4 g to about 7 g citrulline per day.

The nutritional compositions of the present disclosure may also include a synergistic combination of α-HICA and α-ketoglutarate (“AKG”), a precursor of glutamine. In a piglet model where the piglet was stressed with lipopolysaccharide (“LPS”) administration, AKG increased phosphorylation of intestinal mammalian target of rapamycin (“mTOR”) leading to increased protein synthesis and anti-inflammatory responses. Also, AKG increased villous height and reduced crypt depth, and therefore, the potential to increase absorptive capacity (increased absorption of amino acids). Applicant has found that these potential benefits of nutritional compositions including α-HICA and AKG (e.g., oxidative damage, absorption) may lead to increased nutrient delivery leading to further anabolism particularly in inflammatory conditions.

As mentioned above, the nutritional compositions of the present disclosure may be provided to an individual or patient in one bolus, or in several smaller doses. However, the nutritional compositions of the present disclosure should provide the individual with an amount of AKG ranging from about 2 g to about 20 g of α-ketoglutarate per day or from about 10 g to about 30 g per day. The AKG may be in the form of ornithine AKG, arginine AKG, or combinations thereof.

The addition of exogenous nucleotides can make the AKG more effective by two mechanisms: (i) the maintenance of AKG levels by reducing the use of glutamine to make nucleotides in the intestinal tract, and (ii) the enhanced maintenance of villous height as shown in previous studies with nucleotides. The intestinal health provided by nucleotides is especially important in the elderly due to malnutrition or just the reduced general anabolism associated with increased age.

Branched chain amino acids (“BCAA”), are known to be indispensible amino acids. BCAAs, along with other indispensible amino acids, must be provided exogenously to allow for muscle protein synthesis. BCAAs, especially leucine, also serve as signaling molecules to stimulate muscle protein synthesis. This can be via two mechanisms. The first mechanism is stimulation of insulin release since leucine is a strong secretagogue. The second mechanism is more direct as leucine can stimulate the eukaryotic inducing factor that turns on muscle protein synthesis.

It is important to provide all three BCAAs (i.e., leucine, isoleucine, and valine) in any formulation since the large increase of one BCAA can cause a relative deficiency of the other two BCAAs. As BCAAs are known for their undesirable sensory profile, addition of analogs such as α-HICA as well as designer, or high quality, proteins such as, for example, whey protein micelles is a effective way of delivering the benefit while improving patient compliance and therefore clinical outcome leading to better quality of life as well as health economic advantages. Further, combinations with immunomodulating agents such as lactowolfberry can bring synergistic benefits to the patient with low graded inflammation, suppressed anabolism and immunosenescence (e.g., elderly, or those with illness).

In another embodiment, nutritional compositions of the present disclosure may include α-HICA and an ω-3 fatty acid. Example of ω-3 fatty acids include, for example, docosahexaenoic acid (“DHA”), eicosapentaenoic acid (“EPA”) and α-linolenic acid (“ALA”). EPA, an omega-3 polyunsaturated fatty acid, has been shown to attenuate skeletal muscle atrophy in cancer cachexia as well as sepsis and to reduce unloading-induced bone loss through a common cellular signaling pathway by minimizing activation of nuclear factor-κβ (“NF-kβ”). Applicant has found that nutritional compositions having α-HICA and EPA synergistically impact musculoskeletal health through both an attenuated loss of lean body mass and bone mineral density through targeted inhibition of NFkβ. Further, α-HICA and EPA can enhance skeletal muscle protein synthesis (as mediated through the mTOR pathway) and reduce endogenous muscle proteolysis (as mediated through the ubiquitin-proteasome pathway), respectively, under catabolic, disuse or aging conditions. The nutritional therapy will result in preserved lean body mass, which will provide tonic loading to the underlying bone and act as an osteogenic stimulus for bone turnover and minimize fracture risk.

Improved preservation of lean body mass will help to maintain metabolic homeostasis and functional mobility. Further the preservation of bone mass density will reduce the risk of fracture thus leading to improved quality of life as well as healthcare cost savings.

The nutritional compositions of the present disclosure may be provided to an individual or patient in one bolus, or in several smaller doses. However, the nutritional compositions of the present disclosure should provide the individual with an amount of EPA ranging from about 0.25 g to about 5 g, more preferably form about 250 mg to about 3 g, even more preferably from about 250 mg to 1.5 g of eicosapentaenoic acid per day. In an embodiment, the individual is provided with about 750 mg of EPA per day.

The delivery and bioavailability of nutritional compositions having α-HICA and EPA can be improved by (i) packaging (e.g., providing a UV-barrier and/or O₂ scavenging inner layers); (ii) manufacturing (e.g., providing aseptic production, reducing “head space,” and reducing heat exposure), and (iii) encapsulation of a lipid emulsion containing both α-HICA and EPA (e.g., protecting the composition during manufacturing and initial digestion). Further, a vegetarian source of EPA can provide a sustainable source of long-chain polyunsaturated fatty acids (“LC-PUFA”) with improved organoleptic properties.

The nutritional compositions of the present disclosure may provide effective amounts of α-HICA to prevent muscle wasting. Muscle wasting is commonly noted in individuals with chronic kidney disease. Applicant has found, however, that the application of α-HICA to the kidney disease patient segment has several benefits. For example, administering nutritional compositions having α-HICA to the kidney disease patient segment may provide nitrogen or protein sparing effects and improve nitrogen balance in chronic renal failure especially in patients displaying uremia. Branched chain α-keto acids, and α-HICA can take up amine groups from the elevated nitrogenous environment of the uremic patient and thus reduce the overall nitrogenous load. This substitution also partly reduces the total protein intake by patients thereby reducing a further increase in the nitrogen load in uremia patients both of which ameliorate the toxicity associated with elevated urea levels. Providing a portion of the protein needs via substitution with α-HICA and/or other keto-acids may improve the total protein intake of the patient that may support muscle protein.

Further, α-HICA like its precursor leucine, may stimulate muscle protein synthesis and/or limit muscle protein breakdown beneficial for this patient population. U.S. Pat. No. 4,752,619 supports the use of the aforementioned products in conjunction with 20-30 g/day mixed quality protein diet, and a vitamin and mineral supplement.

Applicant has also surprisingly found that nutritional compositions of the present disclosure having a combination of α-HICA and L-carnitine demonstrate synergistic effects in chronic kidney patients, and especially in patients suffering from uremia. L-carnitine is a quaternary ammonium compound biosynthesized from the amino acids lysine and methionine in the liver and kidney. It is found to be deficient in kidney disease owing to impaired biosynthesis, reduced protein intake and losses via dialysis in dialyzed patients. The benefits of L-carnitine supplementation in kidney disease patients may include improvement in erythropoietin-resistant anemia, muscle symptoms, cardiac performance and functional capacities, benefits that may also support muscle function. A combination of α-HICA and L-carnitine will offer the dual benefit of alleviating the uremic load to an extent while providing the deficient product L-carnitine that may support muscle function at least in part, due to its primary function as a transporter of long chain fatty acids to the mitochondria for energy-yielding oxidation.

Existing nutrition support solutions for elderly and patients that have insufficient muscle anabolism and excessive muscle catabolism are lacking in effectiveness. Furthermore, in elderly individuals, there is significant lean body mass loss leading to loss of independence, functionality and quality of life. Further, there is a decline in cognitive ability in elderly patients, and the healthcare costs associated with these morbidities are high. The traditional response to loss of lean body mass has been to provide an increased level of protein to the patient.

Applicant has found that the use of additional beneficial ingredients allows for more efficient use of the administered protein for preservation of lean body mass. Thus, the nutritional compositions of the present disclosure improve the preservation of lean body mass in elderly individuals or patients at risk of muscle loss (e.g. sarcopenia, cachexia, immobilization) by increasing muscle anabolism while simultaneously reducing muscle catabolism. The ingredients that provide the benefit of increased anabolism and decreased catabolism and can be incorporated into both oral supplements and complete feeding products suitable for complete feeding by either oral or tube feeding administration. The nutritional compositions of the present disclosure can also be assembled and packaged as powders for dissolution at the time of use.

In an embodiment wherein the nutritional compositions are oral supplements, the supplements may contain active ingredients plus an appropriate nutritional profile that contains 10 or more grams of high quality protein, which may be provided as whey protein micelle, lipids with the EPA and DHA, and carbohydrates for energy and palatability. Vitamins such as vitamin D and minerals and ingredients such as lactowolfberry, and nucleotides may also be included.

Complete feeding products may have all of the nutrients necessary to support life plus the active ingredients necessary for increased anabolism and decreased catabolism (e.g., α-HICA and/or other beneficial ingredients such as L-carnitine, citrulline, AKG, EPA, etc.).

The nutritional compositions of the present disclosure may be administered by any means suitable for human administration, and in particular for administration in any part of the gastrointestinal tract. Enteral administration, oral administration, and administration through a tube or catheter are all covered by the present disclosure. The nutritional compositions may also be administered by means selected from oral, rectal, sublingual, sublabial, buccal, topical, etc.

If the nutritional compositions are formulated to be administered orally, the compositions may be a liquid oral nutritional supplement (e.g., incomplete feeding) or a complete feeding. In this manner, the nutritional compositions may be administered in any known form including, for example, tablets, capsules, liquids, chewables, soft gels, sachets, powders, syrups, liquid suspensions, emulsions and solutions in convenient dosage forms. In soft capsules, the active ingredients are preferably dissolved or suspended in suitable liquids, such as fatty oils, paraffin oil or liquid polyethylene glycols. Optionally, stabilizers may be added.

Suitable nutritional composition formats according to the present disclosure include, for example, infant formulas, solutions, ready-for-consumption compositions (e.g. ready-to-drink compositions or instant drinks), liquid comestibles, soft drinks, juice, sports drinks, milk drinks, milk-shakes, yogurt drinks, soup, etc. In a further embodiment, the nutritional compositions may be manufactured and sold in the form of a concentrate, a powder, or granules (e.g. effervescent granules), which are diluted with water or other liquid, such as milk or fruit juice, to yield a ready-for-consumption composition (e.g. ready-to-drink compositions or instant drinks).

The nutritional compositions may also include a source of ω-3 and/or ω-6 fatty acids. Examples of sources of ω-3 fatty acids include, for example, fish oil, krill, plant sources of ω-3, flaxseed, walnut, and algae. Non-limiting examples of ω-3 fatty acids include α-linolenic acid (“ALA”), docosahexaenoic acid (“DHA”), and eicosapentaenoic acid (“EPA”). Non-limiting examples of ω-6 fatty acids include linoleic acid (“LA”), arachidonic acid (“ARA”).

In a preferred embodiment, the ω-3 fatty acids are provided in an amount of about 0.25 g to 5.0 g per day, preferably about 1.0 to 3.0 g per day.

In an embodiment, the nutritional compositions include a source of phytochemicals. Phytochemicals are non-nutritive compounds that are found in many fruits and vegetables, among other foods. There are thousands of phytochemicals that can be categorized generally into three main groups. The first group is flavonoids and allied phenolic and polyphenolic compounds. The second group is terpenoids, e.g., carotenoids and plant sterols. The third group is alkaloids and sulfur containing compounds. Phytochemicals are active in the body and, in general, act similarly to antioxidants. They also appear to play beneficial roles in inflammatory processes, clot formation, asthma, and diabetes.

In an embodiment, the nutritional compositions include a source of protein. The protein source may be dietary protein including, but not limited to animal protein (such as milk protein, meat protein or egg protein), vegetable protein (such as soy protein, wheat protein, rice protein, and pea protein), or combinations thereof. In an embodiment, the protein is selected from the group consisting of whey, chicken, corn, caseinate, wheat, flax, soy, carob, pea or combinations thereof.

In an embodiment, vegetable proteins will be included to further enhance the net alkaline profile of the formula and increase the variety of macronutrient sources. Based on the nutritional profile of specific vegetable proteins (e.g., pea protein isolate) there are limitations in the amount of vegetable protein sources that can be included in a formula. For example, the amino acid profile of pea protein includes all of the indispensable amino acids. Pea protein is relatively rich in arginine, but limiting in the sulphur-containing amino acids, methionine, and cysteine. However, it is possible, for example, to blend pea protein isolates with a complete protein source (such as milk protein or complete vegetable proteins) having sufficient sulphur-containing amino acids to offset such deficiency. Canola protein (i.e., isolates, hydrosylates and concentrates) is one such vegetable protein which can provide appreciable amounts of sulfur-containing amino acids to further augment the amino acid profile to deliver the necessary protein quality to the patient. Additionally, animal derived proteins are typically more abundant in sulphur-containing amino acids than vegetable proteins.

The nutritional compositions of the present disclosure may also include a source of carbohydrates. Any suitable carbohydrate may be used in the present nutritional compositions including, but not limited to, sucrose, lactose, glucose, fructose, corn syrup solids, maltodextrin, modified starch, amylose starch, tapioca starch, corn starch or combinations thereof.

The nutritional compositions may also include grains. The grains may include, for example, whole grains, which may be obtained from different sources. The different sources may include semolina, cones, grits, flour and micronized grain (micronized flour), and may originate from a cereal or a pseudo-cereal. In an embodiment, the grain is a hydrolyzed whole grain component. As used herein, a “hydrolyzed whole grain component” is an enzymatically digested whole grain component or a whole grain component digested by using at least an α-amylase, which α-amylase shows no hydrolytic activity towards dietary fibers when in the active state. The hydrolyzed whole grain component may be further digested by the use of a protease, which protease shows no hydrolytic activity towards dietary fibers when in the active state. The hydrolyzed whole grain component may be provided in the form of a liquid, a concentrate, a powder, a juice, a puree, or combinations thereof.

A source of fat may also be included in the present nutritional compositions. The source of fat may include any suitable fat or fat mixture. For example, the fat source may include, but is not limited to, vegetable fat (such as olive oil, corn oil, sunflower oil, high-oleic sunflower, flax seed oil, rapeseed oil, canola oil, high oleic canola oil, hazelnut oil, soy oil, palm oil, coconut oil, blackcurrant seed oil, borage oil, lecithins, and the like), animal fats (such as milk fat), or combinations thereof. The source of fat may also be less refined versions of the fats listed above (e.g., olive oil for polyphenol content).

In an embodiment, the nutritional compositions further include one or more prebiotics. Non-limiting examples of prebiotics include acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, or combinations thereof.

The nutritional compositions may further include one or more probiotics. Non-limiting examples of probiotics include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, non-replicating microorganisms, or combinations thereof.

One or more amino acids may also be present in the nutritional compositions. Non-limiting examples of amino acids include alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, or combinations thereof.

In a preferred embodiment, glutamine is provided in an amount of about 10 g to 40 g per day.

One or more antioxidants may also be present in the nutritional compositions. Non-limiting examples of antioxidants include astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, or combinations thereof.

The nutritional compositions also include fiber or a blend of different types of fiber. The fiber blend may contain a mixture of soluble and insoluble fibers. Soluble fibers may include, for example, fructooligosaccharides, acacia gum, inulin, etc. Insoluble fibers may include, for example, pea outer fiber.

The nutritional compositions of the present disclosure may be a source of either incomplete or complete nutrition. The nutritional compositions may be administered by oral administration or tube feeding. If the nutritional compositions are formulated to be administered orally, the compositions may be a liquid oral nutritional supplement or feeding. The nutritional compositions may also be used for short term or long term tube feeding.

In yet another embodiment, methods of administering the nutritional compositions of the present disclosure are provided. For example, in an embodiment, methods for stimulating muscle protein synthesis in an individual in need of same are provided. In another embodiment, methods for minimizing catabolism of muscle protein in an individual in need of same are provided. In yet another embodiment, methods for preserving lean body mass in an individual in need of same are provided. In still yet another embodiment, methods for reducing unloading-induced bone loss in an individual in need of same are provided. In yet another embodiment, methods for attenuating skeletal muscle atrophy in an individual in need of same are provided. In another embodiment, methods for alleviating a high uremic load in an individual in need of same are provided. The methods include administering to the individual a nutritional composition comprising an effective amount of α-hydroxyisocaproic acid. The nutritional compositions of the present disclosure may also include other active or inactive ingredients as discussed herein above.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

### Example

### Example 1

### Effect of Alpha-HICA on Muscle Atrophy and Recovery after Hindlimb Immobilization in Rats

Materials and Methods

Animal protocols. The experiments described herein were broadly organized into two experimental series. Both studies used male Wistar rats (Charles River Breeding Laboratories, Cambridge, Mass.) which acclimated for 1 week in a controlled environment. Rats were shipped at 350-375 g and were approximately 12 wks of age. Water and standard rat chow were provide ad libitum. All experiments were approved by the Institutional Animal Care and Use Committee of The Pennsylvania State University College of Medicine and adhered to the National Institutes of Health guidelines for the use of experimental animals.

Study 1: This study examined the ability of the various dietary supplements to ameliorate or prevent the normal atrophic response in skeletal muscle produced by disuse atrophy. The following custom diets were commercially prepared (Dytes, Bethlehem, Pa.): control diet (AIN93M), or isocaloric, isonitrogenous diets supplemented with α-hydroxy-isocaproic acid (HICA) or leucine (Leu) (Table 1). A preliminary study indicated the rats had variable consumption of the different diets when they were first introduced. Therefore, animals were provided the dietary intervention for a period of 6 days prior to hindlimb immobilization. After the first day, all animals were pair-fed to the HICA group, which demonstrated the lowest spontaneous food consumption. On day 7, all rats were anesthetized using isoflurane (3% induction+1.5-2% maintenance) and subjected to unilateral hindlimb immobilization via a fiberglass cast, exactly as described (Krawiec B J, Frost R A, Vary T C, Jefferson L S and Lang C H. Hindlimb casting decreases muscle mass in part by proteasome-dependent proteolysis but independent of protein synthesis. Am J Physiol Endocrinol Metab 289: E969-E980, 2005, or Vargas R and Lang C H. Alcohol accelerates loss of muscle and impairs recovery of muscle mass resulting from disuse atrophy. Alcohol Clin Exp Res 32: 128-137, 2008).

The foot was positioned in plantar-flexion to induce maximal atrophy of the gastrocnemius and rats received 10 ml of 0.9% warmed (37° C.) sterile saline for resuscitation. Previous studies have demonstrated that unilateral immobilization has no effect on various parameters of interest in skeletal muscle from the contralateral non-casted leg. Consequently, the contralateral limb served as the control in all subsequent experiments. After casting, rats were housed individually and were continued to be pair-fed for a period of 7 days. Water was provided ad libitum. On day 7 after casting, rats were anesthetized with pentobarbital and the cast atraumatically and rapidly (<2 min) removed (Stryker Instruments, Kalamzoo, Mich.).

Study 2: This study was performed exactly as study 1 above, except that rats were permitted a 7- or 14-day recovery period after cast removal. Because we wished to minimize the duration of anesthesia, animals in this study were anesthetized with isoflurane instead of pentobarbital for cast removal.

TABLE 1
Diet Composition

| | Control | HICA | Leucine |
|---|---|---|---|
| Casein | 166 | 166 | 166 |
| L-Cystine | 1.8 | 1.8 | 1.8 |
| Alanine | 45.3 | 0 | 0 |
| Leucine | 0 | 0 | 50 |
| Valine | 0 | 6 | 6 |
| Isoleucine | 0 | 10 | 10 |
| α-HICA | 0 | 50.4 | 0 |
| Rapeseed oil | 3 | 30 | 30 |
| Sunflower oil | 3 | 3 | 3 |
| Groundnut oil | 27 | 27 | 27 |
| Sucrose | 100 | 100 | 100 |
| Lactose | 134 | 134 | 134 |
| Wheat starch | 412.9 | 391.8 | 392.2 |
| Cellulose | 35 | 35 | 35 |
| AIN 93M mineral Mix | 35 | 35 | 35 |
| AIN93M vitamin mix | 10 | 10 | 10 |
| Total | 1000 | 1000 | 1000 |

All values are g/kg diet.

Analytical Methods.

The in vivo rate of protein synthesis in gastrocnemius, liver and heart (ventricle only) was determined using the flooding-dose technique, exactly as described (Vary T C and Lang C H, Assessing effects of alcohol consumption on protein synthesis in striated muscles. Methods Mol Biol 447: 343-355, 2008). A P-50 catheter was placed in the left carotid artery for the withdrawal of blood. Rats were injected intravenously (IV) with [³H]-L-phenylalanine (Phe; 150 mM, 30 μCi/ml; 1 ml/100 g body weight) and blood was collected 15 min later for determining the plasma Phe concentration and radioactivity. Thereafter, tissues were rapidly excised and a portion freeze-clamped and then stored at −70° C. The rate of protein synthesis was calculated by dividing the amount of radioactivity incorporated into protein by the plasma Phe specific radioactivity. The specific radioactivity of the plasma Phe was measured by high performance liquid chromatography (HPLC) analysis of supernatant from trichloroacetic acid (TCA) extracts of plasma. In addition, samples of fresh muscle were homogenized for Western blot and analysis of selected proteins and another piece of tissue used for qRT-PCR, as described below.

Fresh skeletal muscle was homogenized (Kinematic Polytron; Brinkmann, Westbury, N.Y.) in icecold homogenization buffer consisting of (in mmol/L): 20 HEPES (pH 7.4), 2 EGTA, 50 sodium fluoride, 100 potassium chloride, 0.2 EDTA, 50 β-glycerophosphate, 1 DTT, 0.1 phenylmethane-sulphonylfluoride, 1 benzamidine, and 0.5 sodium vanadate. Protein was determined after centrifugation and equal amounts of protein per sample were subjected to standard SD S-PAGE. Specifically, Western analysis was performed for total and phosphorylated S6K1 (T389; Beverly, Mass.). Blots were developed with enhanced chemiluminescence Western blotting reagents (Supersignal Pico, Pierce Chemical, Rockford, Ill.). Dried blots were exposed to x-ray film to achieve a signal within the linear range and film was then scanned (Microtek ScanMaker IV) and quantified using Scion Image 3b software (Scion Corporation, Frederick, Md.).

RNA Extraction and Real-Time Quantitative PCR

Total RNA was extracted using Tri-reagent (Molecular Research Center, Inc., Cincinnati, Ohio) and RNeasy mini kit (Qiagen, Valencia, Calif.) protocols. Skeletal muscle (50-80 mg) was homogenized in 800 μl of tri-reagent followed by chloroform extraction according to the manufacturer's instruction. Equal volume of 70% ethanol was added to the aqueous phase and the mixture was loaded on a Qiagen mini spin column. The Qiagen mini kit protocol was followed from this step onwards including the on-column DNase I treatment at room temperature to remove residual DNA contamination. RNA was eluted from the column with 40 μl of RNase-free water and 1 μl was used for quantitation on a NanoDrop 2000 (Thermo Fisher Scientific, Waltham, Mass.) spectrophotometer. Quality of the RNA was analyzed on a 1% agarose gel. Total RNA (1 μg) was reversed transcribed using superscript III reverse transcriptase (Invitrogen, Carlsbad, Calif.) following manufacturer's instruction. Real-time quantitative PCR was performed using 25 ng of cDNA in a StepOnePlus system using TaqMan gene expression assays for atrogin (Rn00591730_m1), murf (Rn00590197_m1) ubiquitin b (Rn03062801_gH) and gapdh (Rn01775763_g1) and the gene expression master mix according to the manufacturer's instruction (Applied Biosystems, Foster City, Calif.). The cycling parameters were an initial 95 C for 10 min and 40 cycles of 95° C. for 15 sec and 60° C. for 1 min. The comparative quantitation method 2-ΔΔCt was used in presenting gene expression of target genes in reference to the endogenous control (Livak K J and Schmittgen T D. Analysis of relative gen expression data using real-time quantitative PCR and the 2(−ΔΔC(T)) Method. Methods 25:402-408, 2001).

Blood was collected from the arterial catheter while rats were anesthetized and prior to injection of [³H]-Phe. Blood was dispensed to determine standard hematological and biochemical endpoints. For hematology analyses, 250-500 μl of blood was dispensed to tubes containing EDTA (BD No. 365974; Fisher Scientific). Hematology analyses (Heska CBC-Diff Hematology Analyzer, Loveland, Colo.) included: red and white blood cell counts, hematocrit, hemoglobin, platelets, and differential leukocyte counts. Reticulocytes were counted manually using methylene blue stain. In addition, 900 μl blood was dispensed to a tube containing 100 μl of 0.109 M sodium citrate (BD Medical No. 363083; Fisher Scientific) for measurement of prothrombin time (BBL Fibrometer System, Cockeysville, Md.). The remaining blood was dispensed to a silicone-coated collection tube (BD No. 366381; Fisher Scientific) and allowed to clot. Clotted blood samples were centrifuged in a Beckman Coulter Allegra X-12R centrifuge at 3500 rpm for 5 min at 4° C., and the serum was collected and stored. Biochemical analyses on serum was performed on a Cobas Mira Plus Chemistry Analyzer (Diamond Diagnostics, Holliston, Mass.) and included: total bilirubin, glucose, aspartate aminotransferase, alanine aminotransferase, lactate dehydrogenase, albumin, calcium, creatinine, urea, phosphate, total cholesterol, chloride, and total protein. Serum sodium and potassium were analyzed by flame photometer (IL940; Instrumentation Laboratory, Lexington, Mass.). Triglycerides and free fatty acids (FFAs) were determined colorimetrically (Abcam, Cambridge, Mass.; Wako Diagnostics, Richmond, Va., respectively). Insulin was determined by ELISA (Alpco diagnostics, Salem, N.H.). The homeostatic model assessment of insulin resistance (HOMA-IR) was calculated to provide an estimate of whole-body insulin resistance (Turner R C, Holman R R, Matthews D, Hockaday T D and Peto J. Insulin deficiency and insulin resistance interaction in diabetes: estimation of their relative contribution by feedback analysis from basal plasma insulin and glucose concentrations. Metabolism 28: 1086-1096,1979).

Longitudinal and group differences in body composition were tracked noninvasively in conscious animals using a 1H-NMR analyzer (Bruker LF90 proton-NMR Minispec; Bruker Optics, Woodlands, Tex.) for rapid measurement of total body lean and adipose tissue mass. Measurements were performed immediately prior to cast application and again upon cast removal and/or after a period of recovery.

Statistical analysis. Data for each condition are summarized as means±standard error of the mean (SEM) where the number of rats per treatment group is indicated in the legend to the figure or table. Statistical evaluation of the data was performed using 2-way ANOVA with posthoc Student-Neuman-Keuls test when the interaction was significant. To compare the immobilization-induced decrease in muscle protein synthesis between the right and left gastrocnemius in the same rat, a 2-tailed paired-test was performed. Differences between groups were considered significant at P<0.05.

Results

Food Consumption, Body Weight and Organ Weights.

Animals from all studies were combined to so as to present the overall pattern for food intake and change in body weight during the basal period (e.g., pre-casting), during immobilization and during the recovery period. After day 1, food intake did not differ among the 4 groups until the time of immobilization (e.g., days 2-5; FIG. 1A). Immediately after casting, food intake dropped approximately 25% and the reduction was comparable in all groups. Despite our best attempt to pair-feed rats to the amount of food consumed by rats in the HICA group, the average food consumption of the HICA group tended to be lower than all other groups during the final 4 days of immobilization. Upon cast removal, food consumption gradually increased over the duration of the study, and there was no significant difference in food consumption among the four groups (FIG. 1A).

FIG. 1B illustrates the absolute change in body weight normalized to each animals own starting weight. There was an initial drop in body weight for the first 24 h for all rats upon introduction of the defined diets. Thereafter, body weight increased gradually in all groups and was not different from starting values in any group. As a result of casting, all rats lost body weight and the decrement was comparable in all groups. Upon cast removal, body weight initially increased and then appeared to largely plateau during the recovery period. Overall, there were not sustained differences in either food consumption or body weight among rats receiving the different dietary supplements. Hence, any subsequently described metabolic differences between groups cannot be attributed to differences in caloric intake.

There was no difference in the total organ weight for liver, heart (ventricle only), adrenal gland, spleen or testes (Table 2). Kidney weight did not differ between rats fed the control, HICA or Leucine-enriched diet.

TABLE 2
Organ weights

| | Control | HICA | Leucine |
|---|---|---|---|
| Liver, g | 11.34 ± 0.25 | 12.00 ± 0.35 | 11.49 ± 0.28 |
| Heart, g | 1.11 ± 0.0 | 1.13 ± 0.02 | 1.12 ± 0.01 |
| Kidney, g | 1.19 ± 0.02 | 1.22 ± 0.03 | 1.19 ± 0.02 |
| Adrenal, mg | 380 ± 13 | 403 ± 16 | 398 ± 16 |
| Spleen, mg | 936 ± 32 | 960 ± 37 | 1001 ± 32 |
| Testes, g | 1.84 ± 0.03 | 1.82 ± 0.03 | 1.83 ± 0.04 |

Values are means ± SEM; n = 26, 22 and 23 rats per group, respectively. Data were combined from all studies as there was no difference in organ weights between studies from the same dietary treatment.

In vivo-determined rate of organ protein synthesis was determined between 0800-1000 hours in freely fed rats. Protein synthesis in heart and liver did not differ among the four groups (Table 3).

TABLE 3
In-vivo-determined tissue protein synthesis

| | Control | HICA | Leucine |
|---|---|---|---|
| Heart | 2.96 ± 0.10 | 2.76 ± 0.11 | 2.93 ± 0.11 |
| Liver | 23.62 ± 0.71 | 24.41 ± 0.90 | 22.51 ± 0.88 |
| GAstrocnemius | 1.42 ± 0.04 | 1.26 ± 0.04 | 1.32 0.04 |
| (control) | | | |

Values are means ± SEM; n = 27, 23 and 23 rats per group, respectively. Data were combined from all studies as there was no difference in organ weights between studies from the same dietary treatment.

General Metabolic, Hematological and Organ Characteristics.

Various biochemical endpoints were determined on the serum collected from the four treatment groups. Again, data from all studies were combined because there was no statistical difference detected within groups having consumed the same diet for various durations. As presented in Table 4, although all values were within normal limits for rats, there were some small albeit statistically significant changes among groups for selected endpoints. For example, while no difference was detected for the concentration of several electrolytes (e.g., sodium, chloride and calcium), the serum potassium concentration was lower in the Leu supplemented group, compared to values from either the control- or HICA-fed rats. Also, the serum phosphorus concentration in the Leu group was elevated 18%, compared to control values. Markers of renal function (e.g., creatinine and BUN) were generally unaffected in both the HICA and Leu supplemented groups, compared to control values. Surrogate markers of liver function (AST, ALT, bilrubin) did not differ among groups.

Markers of nutrition and metabolism (total protein, albumin, glucose and triglycerides) also did not differ among the four groups. Finally, the serum insulin concentration did not differ between control and HICA-fed rats (Table 4). Finally, we calculated the HOMA-IR, a surrogate marker of insulin resistance, and demonstrated it to be significantly greater in Leu-fed rats, compared to either control rats or those fed a HICA-containing diet.

TABLE 4
Blood chemistry profile, metabolic substrates and hormone

| | Control | HICA | Leucine |
|---|---|---|---|
| Sodium, | 140 ± 1 | 141 ± 1 | 140 ± 1 |
| mmol/L | | | |
| Potassium, | 4.9 ± 0.1a | 4.9 ± 0.1a | 4.1 ± 0.1b |
| mmol/L | | | |
| Calcium, | 9.5 ± 0.1 | 9.5 ± 0.1 | 9.6 ± 0.1 |
| mg/dL | | | |
| Chloride, | 99 ± 1 | 101 ± 1 | 100 ± 1 |
| mmol/L | | | |
| Phosphorous, | 6.2 ± 0.2a | 5.8 ± 0.2a | 7.0 ± 0.2b |
| mg/dL | | | |
| Creatinine, | 0.3 ± 0.02 | 0.3 ± 0.02 | 0.3 ± 0.01 |
| mg/dL | | | |
| BUN, mg/dL | 16.1 ± 0.6 a | 16.2 ± 0.6a | 16.2 ± 0.5a |
| Bilirubin, | 0.5 ± 0.04 | 0.5 ± 0.04 | 0.3 ± 0.02 |
| total, mg/dL | | | |
| LDH, U/L | 292 ± 30 | 232 ± 23 | 169 ± 21 |
| AST, U/L | 76 ± 5 | 74 ± 4 | 68 ± 4 |
| ALT, U/L | 25 ± 2 | 24 ± 1 | 24 ± 1 |
| Protein, | 4.9 ± 0.1 | 5.0 ± 0.1 | 5.0 ± 0.1 |
| total, g/dL | | | |
| Albumin, g/dL | 3.38 ± 0.04 | 3.31 ± 0.04 | 3.42 ± 0.03 |
| Cholesterol, | 66 ± 3 | 62 ± 3 | 63 ± 4 |
| mg/dL | | | |
| Triglycerides, | 678 ± 45 | 609 ± 51 | 667 ± 52 |
| μmol/L | | | |
| Glucose, | 215 ± 9 | 223 ± 11 | 214 ± 8 |
| mg/dL | | | |
| Insulin, | 1.87 ± 0.14 a | 1.90 ± 0.15 a | 3.64 ± 0.32 c |
| ng/ml | | | |
| HOMA-IR | 25.5 ± 2.8 a | 27.1 ± 3.2 a | 47.7 ± 4.6b |

Values are means ± SEM; n = 26, 22, 23 and 23, respectively, for the 4 dietary groups. Means with different letters (a, b, c) are statistically different (P < 0.05; ANOVA-SNK) within same row. HOMA-IR, homeostatic model assessment of insulin resistance

All hematological endpoints determined were within normal limits for rodents and did not differ among the four groups (Table 5). For example, there was no significant difference in the number of white blood cells (WBCs), red blood cells (RBCs) or platelets among the groups. In addition, there were no differences in the percentage of neutrophils, lymphocytes, monocytes, eosinophiles or reticulocytes among groups. The hematocrit, hemoglobin, mean corpuscular volume (MCV) and mean corpuscular hemoglobin (MCH) also did not differ.

TABLE 5
Hematological profile

| | Control | HICA | Leucine |
|---|---|---|---|
| WBC, 103/μl | 2.3 ± 0.2 | 2.5 ± 0.2 | 2.7 ± 0.2 |
| Neutrophils, % | 25 ± 2 | 24 ± 2 | 23 ± 3 |
| Lymphocytes, % | 72 ± 3 | 70 ± 2 | 73 ± 3 |
| Hematocrit, % | 40 ± 1 | 40 ± 1 | 41 ± 1 |
| MCV, fl | 47 ± 1 | 47 ± 1 | 47 ± 1 |
| RBC, 106/μl | 8.61 ± 0.16 | 8.49 ± 0.31 | 8.98 ± 0.28 |
| Hemoglobin, g/dL | 15.9 ± 0.3 | 15.2 ± 0.5 | 15.7 ± 0.3 |
| MCH, pg | 17.9 ± 0.2 | 17.9 ± 0.1 | 17.9 ± 0.2 |
| Platelets, 103/μl | 1114 ± 37 | 1207 ± 60 | 1206 ± 56 |
| Protime, sec | 18 ± 1 | 19 ± 1 | 18 ± 1 |

Values are means ± SEM; n = 19.17.15 and 15, respectively, for the 4 dietary groups. Abbreviations include: MCV, mean corpuscular volume; MCH, mean corpuscular hemoglobin; WBC, white blood cell, RBC, red blood cell. The percentage of monocytes, eosinophiles and reticulocytes averaged <2% each in all groups and were not affected by dietary supplementation (data not shown). Means with different letters (a, b, c) are statistically different (P < 0.05; ANOVASNK) within same row.

Muscle mass and protein synthesis. The wet weight of the non-casted control gastrocnemius did not differ between groups (FIG. 2A). Immobilization decreased gastrocnemius mass in all groups to a similar extent (control=0.58±0.06 g; HICA=0.61±0.09 g; Leu=0.57±0.08 g). There was no increase in gastrocnemius mass 7 days after removal of the cast in rats consuming the control diet (FIG. 2B). Furthermore, muscle regrowth at this time point was not altered by consumption of aHICA or Leu supplemented diet. However, by recovery day 14, all immobilized muscles had regained some mass (FIG. 2C). At this time point, a reduction in mass of the previously immobilized muscle was still detected in the control- and Leu-fed rats, compared to the contralateral uncasted muscle. In contrast, the weight of the previously casted and uncasted control gastrocnemius in the HICA-fed group did not differ. These group differences are more apparent when the data are expressed as the increment in muscle mass (FIG. 2D).

Protein synthesis in the uncasted muscle from control- and Leu-fed rats did not differ (Table 3). In contrast, the basal rate of protein synthesis in the uncasted muscle from the HICA-fed rats was significantly lower (10%) than the control-fed rats. Casting decreased protein synthesis to the same extent in control- and Leu-fed rats. In contrast, there was no immobilization-induced decrease in muscle protein synthesis in rats fed the HICA-containing diet (FIG. 3A, right panel). After a 7-day recovery period, protein synthesis was increased in the previously immobilized muscle (FIG. 3B). While the increment in protein synthesis tended to be greater in the HICA-fed rats, compared to control-fed rats (0.77±0.17 vs 0.46±0.13 nmol Phe/h/mg protein, respectively), this difference did not achieve statistical significance. By day 14 of recovery, the rate of protein synthesis in the previously immobilized leg was not different from the uncasted muscle for the control and Leu groups (FIG. 3C). However, protein synthesis in the previously immobilized muscle of the HICA-fed group was still increased, compared to the contralateral control muscle. Again, the increment in muscle protein synthesis tended to be increased in the HICA-fed rats, compared to other groups, but this change failed to achieve statistical significance (FIG. 3C, right panel).

We simultaneously determined gastrocnemius weight and protein synthesis on a set (n=7) of pair-fed naive rats provided the control diet for the same period of time. The weight of left and right gastrocnemius from this control naive group (2.17±0.04 g and 2.18±0.05 g, respectively) did not differ from the weight of the uncasted control muscle in the control-fed group (2.16±0.05 g). Furthermore, protein synthesis in the left (1.31±0.07 nmol Phe/h/mg protein) and right (1.39±0.08 nmol Phe/h/mg protein) gastrocnemius from naive control rats did not differ from the uncasted muscle in control-fed rats (1.36±0.07 nmol Phe/h/mg protein). These data and those previously published (28) suggest that the contralateral muscle in casted rats is an appropriate internal control and does not undergo significant compensatory hypertrophy.

S6K1 Phosphorylation.

Alterations in mTOR kinase activity typically lead to coordinate changes in the phosphorylation state of its down stream substrates, S6K1, which are proportional to changes in global rates of protein synthesis. The basal level of S6K1 phosphorylation (T389) in the uncasted control muscle showed considerable variability and hence there was no statistical differences among the various treatment groups (FIG. 4). However, immobilization resulted in a consistent reduction in S6K1 phosphorylation which did not differ between groups. Conversely, during the recovery period, S6K1 phosphorylation in the previously immobilized muscle was either elevated (day 7 recovery) or had returned to control values (day 14 recovery).

Discussion

Effect of HICA- and Leu Supplementation on Basal Condition.

The present investigation assessed the ability of diets supplemented with either aHICA or Leu to ameliorate the atrophic response produced by immobilization and/or to improve the ability of muscle to recovery mass after cast removal. The feeding of these different diets for up to 3 wks produced few statistical differences for numerous biochemical and hematological endpoints, compared to rats fed the control diet. Furthermore, weights of various organs (e.g., liver, heart, spleen, kidney, testes) were largely unaffected. Hence, these various dietary supplements do not appear to have any overt organ toxicity.

The feeding of these diets also did not significantly alter the amount or percentage of LBM in rats or the mass of the gastrocnemius. Furthermore, no significant change in the rate of in vivodetermined protein synthesis for liver or heart was detected.

Atrophic Response to Immobilization.

Various models have been used to investigate disuse atrophy, including hindlimb suspension (e.g., unloading), extended bed rest, denervation, and hindlimb immobilization—the latter being either uni- or bi-lateral. While each model has advantages and disadvantages, relative to the others, the present study used unilateral casting to produce disuse atrophy. This model allows comparison of immobilized to control muscle in the same rat, maintains neural innervation to the hindlimb musculature, and permits recovery-type studies to be perform after cast removal. It is also, arguably, more clinically relevant than other models. Our studies were also conducted in approximately 14 wk-old Wistar rats which were no longer in the rapid growth phase of their development. Hence, differences between the uncasted and casted muscle are more likely to represent an atrophic response as opposed to a failure of normal muscle growth. In general, limb immobilization has been reported to decrease muscle mass and fiber diameter in mice, rats and humans. This disuse atrophy is caused by an imbalance between rates of protein synthesis and degradation. The majority of evidence supports both a reduction in mixed or global muscle protein synthesis which commences as early as 6 h after immobilization and is maintained reduced for several days to weeks. In other catabolic conditions characterized by muscle wasting (e.g., sepsis, alcohol, excess glucocorticoids, inflammatory cytokine excess), such a reduction in muscle protein synthesis is temporally associated with a suppression in mTOR activity, as evidenced by a reduction in the phosphorylation of S6K1 ((Kazi A A, Pruznak A M, Frost R A and Lang C H Sepsis-induced alterations in protein-protein interactions within mTOR complex 1 and the modulating effect of leucine on muscle protein synthesis. Shock 35: 117-125, 2011). Our current data demonstrate a clear decrease in S6K1 phosphorylation in response to disuse.

Impact of Dietary Supplementation with aHICA or Leu on the Atrophic Response.

Leucine stimulates global protein synthesis in skeletal muscle predominantly by enhancing mRNA translation initiation (Dennis M D, Baum J I, Kimball S R and Jefferson L S. Mechanisms involved in the coordinate regulation of mTORC1 by insulin and amino acids. J Biol Chem 286: 8287-8296, 2011.9)

However, feeding rats a diet supplemented with Leu alone failed to prevent the casting-induced decrease in protein synthesis in gastrocnemius. Contrary to the lack of Leu effect, feeding rats a diet supplemented with αHICA alone prevented the casting-induced decrease in muscle protein synthesis.

Importantly, despite the ability of αHICA to prevent the normal reduction in muscle protein synthesis, this metabolite failed to prevent or ameliorate the accompanying reduction in muscle mass per se. In this regard, none of the dietary supplements were able to slow the loss of muscle mass produced by disuse.

Impact of Dietary Supplementation with αHICA or Leu on Recovery from Immobilization.

Seven days after cast removal (i.e., “recovery”), protein synthesis was increased in the previously immobilized muscle. Such a compensatory increase in muscle protein synthesis has been previously reported to start as early as 6-24 hours after cast removal. Moreover, the increase in protein synthesis is consistent with the enhanced phosphorylation of S6K1.

By recovery day 14, the compensatory increase in protein synthesis detected in the previously immobilized muscle at 7 days had returned to basal values in the control-, and Leufed rats. However, protein synthesis was still increased in the previously immobilized muscle from rats supplemented with HICA alone. This elevation in protein synthesis was associated with an coordinate increase in the mass of the previously immobilized muscle back to control levels. The complete recovery of mass in the atrophic limb differed from the partial recovery of gastrocnemius mass in rats provide the Leu-enriched diets.

In summary, of the dietary supplements assessed, only αHICA prevent the immobilizationinduced reduction in muscle protein synthesis. Collectively, none of the supplements prevented or ameliorated the reduction in muscle mass produced by unilateral hindlimb immobilization in adults rats. In contrast, αHICA provide throughout the period of immobilization and then for 2 weeks of recovery did produce a sustained increase in muscle protein synthesis in the previously immobilized muscle and a greater increment in muscle mass per se. A similar therapeutic effect on muscle recovery was not seen in rats receiving diets containing supplemental Leu. As there were no apparent deleterious effects of αHICA supplementation on numerous whole-body and tissue-specific metabolic and hematological parameters, our data suggest that provision of αHICA may represent an important nutraceutical approach aimed at speeding recovery from disuse atrophy.

### FIGURE LEGENDS

FIG. 1: Daily food consumption and change in body weight in rats fed a diet enriched in αHICA, or leucine (Leu), compared to pair-fed control rats. The initial absolute body weight for rats in the control, HICA and Leu groups is 398±5, 398±5, 400±5 and 397±6 g, respectively. Values are means±SEM; n=27, 25, 23, and 23, respectively, during days 1-13, and n=19, 15, 15 and 15, respectively, during the first 7 days of recovery and n=8 per group for the 14 day recovery period.

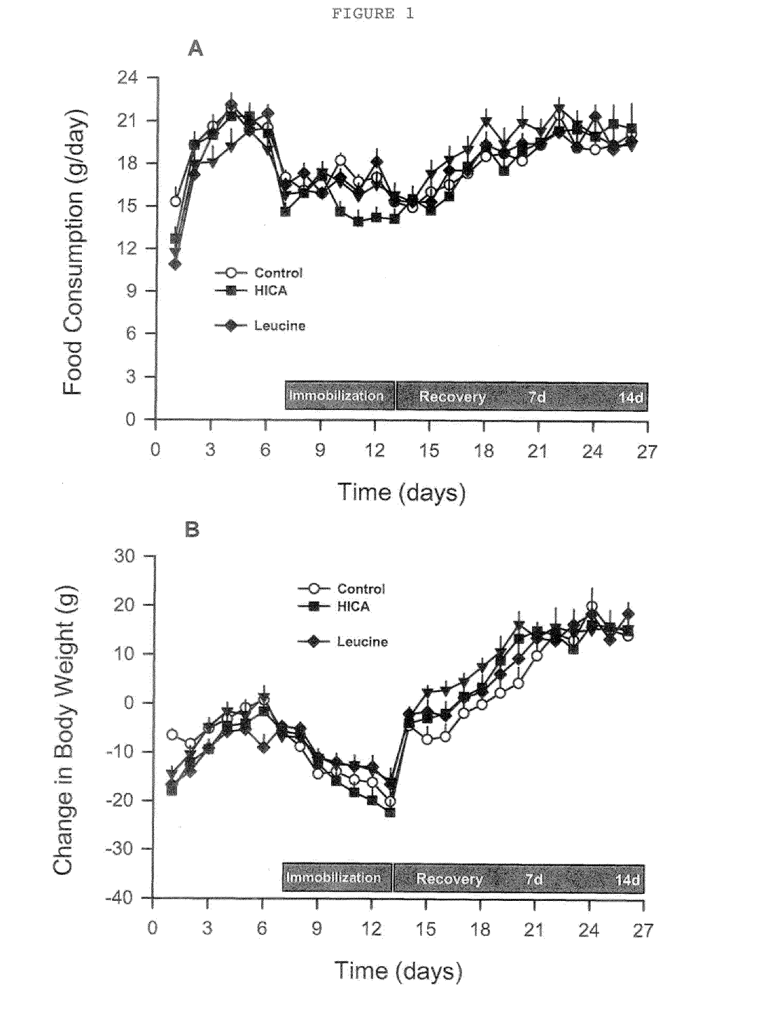

FIG. 2: Weight of gastrocnemius in the un-casted (control) limb or the contralateral limb either at the end of 7 days of immobilization (panel A), or after 7 days of immobilization+7 days of recovery (panel B) or after 14 days of recovery (panel C). Values are means±SEM. The sample size for the control, HICA and Leu groups is n=8, 10, 8 and 9, respectively, for the immobilization period, n=11, 7, 7 and 7 for the 7-day recovery period, and n=8 per group for the 14-day recovery period. *P

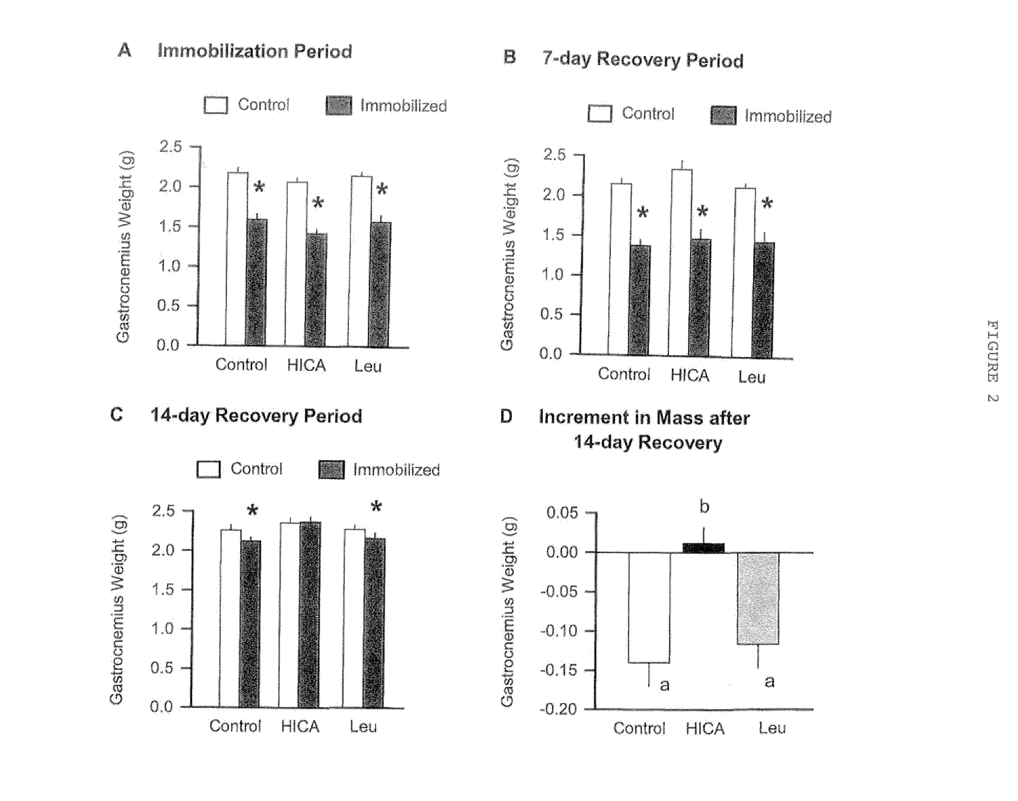

<0.05 compared to un-casted control muscle from the same group at the same time point. The weight of the un-casted muscle did not differ among the different dietary groups for any of the three time points.

FIG. 3: In vivo-determined protein synthesis in gastrocnemius in the un-casted limb or the contralateral limb at the end of 7 days of immobilization (panel A), or after 7 days of immobilization+7 days of recovery (panel B) or after 14 days of recovery (panel C). Values are means±SEM. The sample size for the control, HICA and Leu groups is n=8, 10, 8 and 9, respectively, for the immobilization period, n=11, 7, 7 and 7 for the 7-day recovery period, and n=8 per group for the 14-day recovery period. Left panels represent quantitation of absolute rates of protein muscle protein synthesis, which right panels represent the change (increment or decrement) in protein synthesis (control−casted) in the same animals. *P<0.05 compared to un-casted control muscle from the same group. There was no difference in the weight of the un-casted muscle among the different dietary groups. Values with different letters are significantly different, P<0.05.

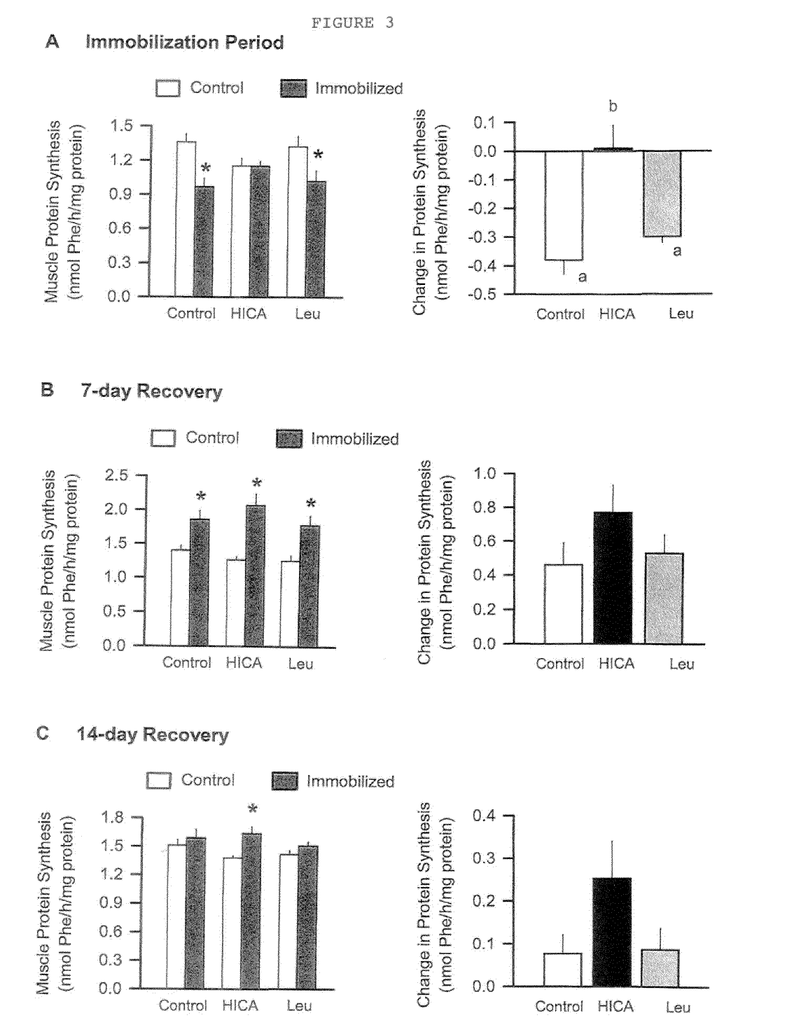

FIG. 4: S6K1 phosphorylation in gastrocnemius in the un-casted limb or the contralateral limb at the end of 7 days of immobilization (panel A), or after 7 days of immobilization+7 days of recovery (panel B) or after 14 days of recovery (panel C). Values are means±SEM. The sample size for the control, HICA and Leu groups is n=8, 10, 8 and 9, respectively, for the immobilization period, n=11, 7, 7 and 7 for the 7-day recovery period, and n=8 per group for the 14-day recovery period. Western blots for each panel are representative of all samples, with samples from each time point having been run on the same gel. Bar graphs are densitometric quantitation of all immunoblots where bars represent means±SEM *P<0.05 compared to un-casted control muscle from the same group. Values with different letters are significantly different, P<0.05.

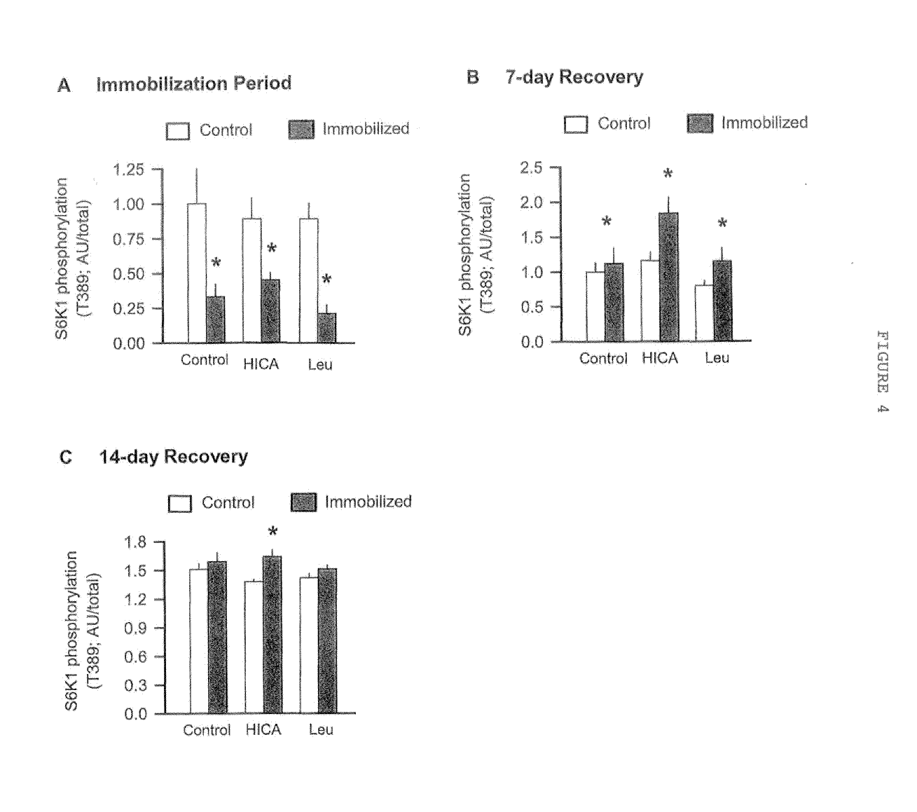

1. A method for achieving a result selected from the group consisting of:
stimulating muscle protein synthesis in an individual in need of same,
minimizing catabolism of muscle protein in an individual in need of same,
preserving lean body mass in an individual in need of same,
reducing unloading-induced bone loss in an individual in need of same,
attenuating skeletal muscle atrophy in an individual in need of same, and
alleviating a high uremic load in an individual in need of same comprising administering a nutritional composition comprising an effective amount of α-hydroxyisocaproic acid to the individual.

2. The method according to claim 1, wherein the individual is selected from the group consisting of the elderly, those with a medical condition, and combinations thereof.

3. The method according to claim 2, wherein the elderly includes those at risk of disability due to sarcopenia, frailty.

4. The method according to claim 1, wherein the nutritional composition is administered to the individual so as to provide the individual with about 150 mg to about 2.5 g of α-hydroxyisocaproic acid per day.

5. The method according to claim 1, wherein the nutritional composition is administered to the individual so as to provide the individual with about 0.15 g to about 10 g of α-hydroxyisocaproic acid per day.

6. The method according to claim 1, wherein the nutritional composition is administered to the individual so as to provide the individual with about 150 mg to about 2.5 g of α-hydroxyisocaproic acid per day.

7. The method according to claim 1, wherein the composition comprises a source of ω-3 fatty acids, wherein the source of ω-3 fatty acids is selected from the group consisting of fish oil, hill, plant sources containing ω-3 fatty acids, flaxseed, walnut, algae, and combinations thereof.

8. The method according to claim 7, wherein the ω-3 fatty acids are selected from the group consisting of α-linolenic acid (“ALA”), docosahexaenoic acid (“DHA”), stearidonic acid (“SDA”) and combinations thereof.

9. The method according to claim 7, wherein the ω-3 fatty acids are provided in an amount of about 0.25 g to 5.0 g per day.

10. The method according to claim 1, wherein the composition comprises a phytonutrient selected from the group consisting of flavanoids, allied phenolic compounds, polyphenolic compounds, terpenoids, alkaloids, sulphur-containing compounds, and combinations thereof.

11. The method according to claim 10, wherein the phytonutrient is selected from the group consisting of carotenoids, plant sterols, quercetin, curcumin, limonin, and combinations thereof.

12. The method according to claim 1, wherein the composition includes a source of protein.

13. The method according to claim 12, wherein the source of protein provides the nutritional composition with at least 10 g of high quality protein.

14. The method according to claim 12, wherein the source of protein provides an individual with at least 10 g of high quality protein per day.

15. The method according to claim 12, wherein the source of protein is selected from the group consisting of dairy based proteins, plant based proteins, animal based proteins, artificial proteins, and combinations thereof.

16. The method according to claim 15, wherein the dairy based proteins are selected from the group consisting of casein, micellar casein, caseinates, casein hydrolysate, whey, whey protein micelles, whey hydrolysates, whey concentrates, whey isolates, milk protein concentrate, milk protein isolate, and combinations thereof.

17. The method according to claim 15, wherein the plant based proteins are selected from the group consisting of soy protein, pea protein, canola protein, wheat and fractionated wheat proteins, corn proteins, zein proteins, rice proteins, oat proteins, potato proteins, peanut proteins, green pea powder, green bean powder, spirulina, proteins derived from vegetables, beans, buckwheat, lentils, pulses, single cell proteins, and combinations thereof.

18. The method according to claim 1, wherein the composition comprises a prebiotic selected from the group consisting of acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, and combinations thereof.

19. The method according to claim 1, wherein the composition comprises a probiotic selected from the group consisting of Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, non-replicating microorganisms, and combinations thereof.

20. The method according to claim 1, wherein the composition comprises an amino acid selected from the group consisting of alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, and combinations thereof.

21. The method according to claim 20, wherein the amino acid is a branched chain amino acid selected from the group consisting of isoleucine, leucine, valine, and combinations thereof.

22. The method according to claim 1, wherein the composition comprises an antioxidant selected from the group consisting of astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, and combinations thereof.

23. The method according to claim 1, wherein the composition comprises a vitamin selected from the group consisting of vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e., MK-4, MK-7), folic acid, biotin, choline and combinations thereof.

24. The method according to claim 1, wherein the composition comprises a mineral selected from the group consisting of boron, calcium, chromium, copper, iodine, iron, magnesium, manganese, molybdenum, nickel, phosphorus, potassium, selenium, silicon, tin, vanadium, zinc, and combinations thereof.

25. The method according to claim 1, wherein the composition includes L-carnitine.

26. The method according to claim 1, wherein the nutritional composition further includes at least one nucleotide selected from the group consisting of a subunit of deoxyribonucleic acid (“DNA”), a subunit of ribonucleic acid (“RNA”), polymeric forms of DNA and RNA, yeast RNA, and combinations thereof.

27. The nutritional composition according to claim 26, wherein the at least one nucleotide is an exogenous nucleotide.

28. The nutritional composition according to claim 26, wherein the nucleotide is provided in an amount of about 0.5 g to 3 g per day.

29. The method according to claim 1, wherein the nutritional composition is in a form selected from the group consisting of tablets, capsules, liquids, chewables, soft gels, sachets, powders, syrups, liquid suspensions, emulsions, solutions, and combinations thereof.

30. The method according to claim 29, wherein the nutritional composition is in the form of a powder.

31. The method according to claim 1, wherein the nutritional composition is an oral nutritional supplement or in a tube feeding

32. The method according to claim 1, wherein the nutritional composition is a source of complete nutrition or of incomplete nutrition.

33. A method for stimulating muscle protein synthesis in an individual in need of same, the method comprising the steps of:
administering to the individual a nutritional composition comprising an effective amount of α-hydroxyisocaproic acid.

34. A method for minimizing catabolism of muscle protein in an individual in need of same, the method comprising the steps of:
administering to the individual a nutritional composition comprising an effective amount of α-hydroxyisocaproic acid.

35. A method for preserving lean body mass in an individual in need of same, the method comprising the steps of:
administering to the individual a nutritional composition comprising an effective amount of α-hydroxyisocaproic acid.

36. A method for reducing unloading-induced bone loss in an individual in need of same, the method comprising the steps of:
administering to the individual a nutritional composition comprising an effective amount of α-hydroxyisocaproic acid.

37. A method for attenuating skeletal muscle atrophy in an individual in need of same, the method comprising the steps of:
administering to the individual a nutritional composition comprising an effective amount of α-hydroxyisocaproic acid.

38. A method for alleviating a high uremic load in an individual in need of same, the method comprising the steps of:
administering to the individual a nutritional composition comprising an effective amount of α-hydroxyisocaproic acid

39. (canceled)
